(19) United States
(12) Patent Application Publication
(10) Pub. No.: US 2026/0048214 A1
(43) Pub. Date: Feb. 19, 2026

(54) SYSTEM FOR CONTROLLING A VENTILATION VARIABLE OF A VENTILATOR AND VENTILATOR

(71) Applicant: HAMILTON MEDICAL AG, Bonaduz (CH)

(72) Inventor: Kevin Meier, Chur (CH)

(21) Appl. No.: 17/631,187

(22) PCT Filed: Jul. 29, 2020
(86) PCT No.: PCT/EP2020/071397

§ 371 Date: Jun. 3, 2025

(30) Foreign Application Priority Data
Jul. 30, 2019 (DE) 10 2019 120 541.7

(51) Int. Cl. A61M 16/00

(52) U.S. Cl. CPC A61M 16/00; A61M 16/024; A61M 16/026

(57) ABSTRACT

A system for controlling a ventilation variable of a ventilation device for tracking a target curve includes at least one sensor to detect instantaneous values of the ventilation variable; an actuator to adjust the ventilation variable of the ventilation device; a memory for storing the target curve for the ventilation variable for an inspiration and/or expiration phase; and a controller having a proportional term and an integral term and that calculates a control difference between the target curve and the instantaneous values, and that controls the actuator accordingly. The controller adjusts the gain factor of the integral term: (i) increasing it when the integral of the control difference for the inspiration and/or expiration phase exceeds a predetermined integral threshold value, or (ii) reducing it when a number of zero crossings of the control difference during the inspiration and/or expiration phase exceeds a predetermined zero-crossing threshold value.

### CROSS-REFERENCE TO RELATED APPLICATIONS

This application is a national phase entry under 35 U.S.C. § 371 of International Application PCT/EP2020/071397, filed Nov. 13, 2019, which claims priority to German patent application Ser. No. 102019120541.7, filed Jul. 30, 2019, all of which are herein incorporated by reference in their entirety.

### FIELD OF THE INVENTION

The present invention is in the field of ventilation devices, i.e. the present invention is in the field of mechanical ventilation of patients. In particular, the present invention relates to the control of a ventilation variable, such as the ventilation pressure or ventilation volume, of a ventilation device.

### BACKGROUND OF THE INVENTION

In mechanical ventilation, breathing air is supplied to a patient by mechanical means, usually by applying a positive pressure. Mechanical ventilation can support or completely replace the patient's own breathing. Ventilation air is supplied in successive ventilation cycles. Each ventilation cycle has an inspiration phase followed by an expiration phase. The ventilation air is supplied during the inspiration phase of a respective ventilation cycle, usually mechanically or in any case mechanically assisted, and at a positive pressure relative to the pressure prevailing in the patient's airways. Exhalation takes place during a subsequent expiration phase of the ventilation cycle. In this phase, there is usually no application of positive or negative pressure to the airways, rather, exhalation usually occurs passively by relaxing the airways relative to the ambient pressure. However, it is also possible for mechanical support to be provided during the expiration phase.

In mechanical ventilation, attempts are often made to apply a ventilation variable, such as the ventilation pressure or the ventilation flow, i.e. the ventilation volume per unit of time, in a time-variable manner. In particular, attempts are made to apply the ventilation variable according to a desired target curve for an inspiration phase and/or for an expiration phase. The desired target curve can be a model for the patient's normal respiratory behavior or can represent a respiratory behavior that is particularly favorable for the patient's current condition, etc.

Applying the ventilation variable according to a desired target curve is generally quite difficult. Previous approaches for applying the ventilation variable according to a desired target curve are not always satisfactory. In particular, the results of previous approaches may diverge significantly for different patients.

Accordingly, it would be desirable to provide a system and a method for controlling a ventilation variable of a ventilation device that allow a wide range of applications for mechanical ventilation. In particular, it would be desirable to provide a system and a method that produce good ventilation results even in the presence of divergent anatomical conditions of patients.

### SUMMARY OF THE INVENTION

Exemplary embodiments of the invention include a system for controlling a ventilation variable of a ventilation device for tracking a target curve, comprising: at least one sensor which is adapted to detect instantaneous values for the ventilation variable; an actuator which is adapted to adjust the ventilation variable of the ventilation device; a memory in which the target curve for the ventilation variable is stored, the target curve being stored for an inspiration phase and/or for an expiration phase; and a controller which has at least a proportional term and an integral term and which is adapted to determine a control difference from the target curve and the instantaneous values for the ventilation variable, and which is adapted to control the actuator; wherein the controller is adapted to adjust a gain factor of the integral term according to at least one of the following features: (i) increasing the gain factor of the integral term when the integral of the control difference for the inspiration phase and/or the expiration phase exceeds a predetermined integral threshold value; (ii) reducing the gain factor of the integral term when a number of zero crossings of the control difference during the inspiration phase and/or during the expiration phase exceeds a predetermined zero-crossing threshold value.

Exemplary embodiments of the invention allow an effective and easy-to-implement adjustment of the control of the respiratory variable for the present ventilation conditions, such as for a patient to be ventilated. Providing a controller having at least a proportional term and an integral term, and adjusting the gain factor of the integral term in the manner described above, allow an effective adjustment of the controller and thus an effective tracking of the ventilation variable along the target curve. In doing so, the controller can adjust its behavior on its own and does not need to rely on data regarding the patient's anatomy. Compared to previous approaches in which the resistance and expansion capacity of the patient's ventilation tract were used to parameterize the controller, exemplary embodiments of the invention allow for an automated adjustment of tracking the target curve during the ventilation. Thus, determination of the resistance and expansion capacity of the patient's ventilation tract may be rendered superfluous by the control system described herein. The inherent problems of such determination of the resistance and expansion capacity of the patient's ventilation tract, namely high complexity and low accuracy of the determination of said parameters, can thus be avoided as well. Exemplary embodiments of the invention allow a control of the ventilation variable so as to permit effective tracking along the target curve, without the need to determine or model in advance patient data that are difficult to determine.

The controller is adapted to increase the gain factor of the integral term when the integral of the control difference for the inspiration phase and/or the expiration phase exceeds a predetermined integral threshold value, and/or to decrease the gain factor of the integral term when a number of zero crossings of the control difference during the inspiration phase and/or during the expiration phase exceeds a predetermined zero-crossing threshold value. In other words, it is possible that the controller is only adapted to increase the gain factor of the integral term when the integral of the control difference for the inspiration phase and/or the expiration phase exceeds a predetermined integral threshold value, or that the controller is only adapted to reduce the gain factor of the integral term when a number of zero crossings of the control difference during the inspiration phase and/or during the expiration phase exceeds a predetermined zero-crossing threshold value, or that the controller is adapted to increase the gain factor of the integral term when the integral of the control difference for the inspiration phase and/or the expiration phase exceeds a predetermined integral threshold value and to decrease the gain factor of the integral term when a number of zero crossings of the control difference during the inspiration phase and/or during the expiration phase exceeds a predetermined zero-crossing threshold value. By increasing the gain factor of the integral term, the controller can be adjusted so as to respond more strongly and thus more quickly to deviations between the ventilation variable and the target curve, in particular to deviations between the ventilation variable and the target curve that extend over a comparatively longer period of time. Reducing the gain factor of the integral term can slow down the controller and thus reduce or prevent overshooting of the target curve, in particular strong oscillation of the ventilation variable around the target curve.

The target curve is stored in the memory for an inspiration phase and/or for an expiration phase. In other words, it is possible that the stored target curve only concerns the inspiration phase or that the stored target curve only concerns the expiration phase or that the stored target curve concerns both the inspiration phase and the expiration phase. It is possible for the controller to track the ventilation variable along a target curve only in the inspiration phase, or to track it along a target curve only in the expiration phase, or to track it along a target curve both in the inspiration phase and in the expiration phase. The controller may have different parameters for the proportional term and the integral term for the inspiration phase and the expiration phase. In other words, the controller may treat the inspiration phase and the expiration phase differently. In particular, the gain factor of the integral term may be different for the inspiration phase and the expiration phase. However, it is also possible that the controller treats the inspiration phase and the expiration phase the same, i.e. that it is parameterized in the same way for the inspiration phase and the expiration phase.

According to a further embodiment, the ventilation variable is one of ventilation pressure and ventilation volume. In other words, the controlled ventilation variable is the ventilation pressure provided by the ventilation device or the ventilation volume provided by the ventilation device, i.e., the volume of air provided by the ventilation device. The ventilation volume may be considered as ventilation volume per unit of time and thus as ventilation flow or air flow. The at least one sensor may comprise at least one pressure sensor and/or at least one volume or flow sensor.

According to a further embodiment, the actuator comprises a controlled blower and/or a controlled valve. In this way, the ventilation variable can be controlled by generating compressed air via a controlled blower and/or by regulating compressed air via a controlled valve. It is possible to control/regulate the ventilation variable solely through an adjustable source, such as a controlled blower, or through an adjustable access to a non-adjustable source, such as e.g. a controlled valve to an air pressure reservoir, or through a combination of an adjustable source and an adjustable access. In particular, for the inspiration phase, the actuator may have only a controlled blower or only a controlled inspiration valve, or a combination of a controlled blower and a controlled inspiration valve. For the expiration phase, the actuator may have a controlled expiration valve. However, for the expiration phase, it is also possible that the ventilation device is a completely passive system through which air is passively released from the patient's lungs as a result of the positive pressure built up in the lungs during the inspiration phase. The actuator may include any component or any combination of components suitable to adjust a desired ventilation variable of the ventilation device.

According to a further embodiment, the controller is a proportional integral controller (PI controller). In other words, the controller has only a proportional term and an integral term. Accordingly, it is possible to design the controller without further elements, e.g. without a differential element and/or without a dead time element. In this way, effective tracking along the target curve is possible with low complexity and high processing speed. A particularly good response behavior can be achieved compared to the typical duration of an inspiration phase or expiration phase.

According to a further embodiment, the controller is adapted to leave a gain factor of the proportional term unchanged when the gain factor of the integral term is adjusted. The inventors of the present system for controlling a ventilation variable of a ventilation device have found that, surprisingly, effective tracking of the target curve can be achieved solely by adjusting the integral term during ventilation. In particular, adjustment of the integral term can be performed without adjustment of the proportional term. In this way, the controller can be adjusted in a low-complexity, easy-to-implement and easy-to-follow manner. The high transparency of the adjustment of the controller is also welcome in light of the fact that ventilating a patient generally constitutes a high risk due to the high sensitivity of the respiratory tract and that tightly constrained and easily reproducible adjustments of the controller in case of irregularities on the part of the patient can contribute to effective troubleshooting.

According to a further embodiment, the controller is adapted to leave the gain factor of the proportional term generally unchanged during operation. In other words, the gain factor of the proportional term may be set to a predetermined value and remain there. In this regard, the gain factor of the proportional term may be the same for all patients or may be set to a patient-specific gain factor at the start of ventilation.

According to a further embodiment, the controller is adapted to set the gain factor of the integral term to an initial value. In this way, control of the ventilation variable can begin in a well-defined manner and be adjusted from a well-defined starting point during ventilation to suit the conditions at hand. The initial value can further ensure that in any case at the beginning there is no parameterization of the integral term present that is potentially dangerous for the patient.

According to a further embodiment, the initial value is dependent on at least one patient parameter, such as e.g. age or weight. In particular, the initial value may depend on readily available macroscopic parameters of the patient. Compared to the resistance and expansion capability of the patient's ventilation tract, patient parameters such as age or weight are easy to determine. Thus, the controller can tailor the control to a specific class of patients without much complexity. For example, the controller can be adjusted for the situation whether the patient is a child or an adult. Furthermore, it is possible to make an initial rough estimate regarding the lung volume based on the weight of the patient, which may be reflected in the initial value.

According to a further embodiment, the controller is adapted to set the gain factor of the integral term to the initial value once at the start of operation. It is also possible to set the gain factor of the integral term to the initial value in predetermined intervals. In this way, the well-defined starting point for the control can be taken once or repeatedly. When setting the gain factor of the integral term to the initial value once, an achieved adjustment of the controller can be maintained for a long time. Repeatedly setting the gain factor of the integral term to the initial value can prevent an erroneous, unwanted development of the gain factor of the integral term from persisting for a long time. The repeated setting of the gain factor of the integral term to the initial value can be used in particular when the adjustment of the gain factor of the integral term is performed in only one direction. In other words, repeatedly setting the gain factor of the integral term to the initial value can be used in particular when the controller is only designed to increase the gain factor of the integral term when the integral of the control difference for the inspiration phase and/or the expiration phase exceeds a predetermined integral threshold value, or when the controller is only designed to decrease the gain factor of the integral term when a number of zero crossings of the control difference during the inspiration phase and/or during the expiration phase exceeds a predetermined zero-crossing threshold value.

According to a further embodiment, the controller is adapted to adjust the gain factor of the integral term for tracking the target curve for the current inspiration phase or the current expiration phase. In other words, the controller may be adapted to adjust the gain factor of the integral term before the current inspiration phase or the current expiration phase ends. In this way, the tracking of the ventilation variable along the target curve can be adjusted immediately for an ongoing inspiration phase or expiration phase. The gain factor of the integral term and thus the parameterization of the controller can be adjusted so quickly that the effect of the adjustment is already noticeable in the current inspiration phase or expiration phase. In particular, the gain factor of the integral term can be immediately increased and/or decreased as soon as one of the conditions described herein for increasing and/or decreasing the gain factor is present. Further in particular, adjusting the gain factor of the integral term upon detecting one of the conditions described herein may occur in a time frame that is much smaller than the duration of an inspiration phase or an expiration phase. Said time frame may be less than 1/10 or less than 1/50 or less than 1/100 of the duration of the inspiration phase or the expiration phase, respectively.

According to an alternative embodiment, the controller is adapted to adjust the gain factor of the integral term for tracking the target curve for the next inspiration phase or the next expiration phase. In this way, the ventilation variable is controlled with a fixed gain factor of the integral term for a given inspiration phase or expiration phase. This, in turn, may allow for a particularly stable, though possibly slower, convergence of the control from ventilation cycle to ventilation cycle.

As described above, the controller may be adapted to increase the gain factor of the integral term when the integral of the control difference for the inspiration phase and/or the expiration phase exceeds the predetermined integral threshold value.

According to a further embodiment, the controller is adapted to set the integral of the control difference to zero when the control difference crosses zero. In this way, increasing the gain factor of the integral term is aimed at considering the integral of the control difference until the control target is reached, i.e. until the target curve is reached. In particular, the integral of the control difference may be considered between successive instances of reaching the target curve. Thus, the integral of the control difference can serve as a good measure of the speed of the controller in tracking the target curve, with the repeated resetting of the integral to zero permitting a repeated assessment of the speed of the controller.

According to a further embodiment, a plurality of integral threshold values are provided, and the controller is adapted to increase the gain factor of the integral term a plurality of times when the integral of the control difference exceeds several of the plurality of integral threshold values. In this manner, multiple degrees of adjustment of the controller can be provided so that the gain factor of the integral term can be particularly well aligned with the control difference or can be adjusted in a particularly targeted manner. Also, in this way, an effective adjustment of the control can be achieved for obtaining a desired tracking along the target curve in particularly fast manner. It is possible for the controller to be adjusted several times during an inspiration phase or an expiration phase, thus directly counteracting an accumulating control error. The plurality of integral threshold values may be multiples of a first integral threshold value. It is also possible for the integral threshold values to be provided in a non-linear manner, for example based on an experimental determination of appropriate integral threshold values. Similarly, the multiple increases of the gain factor of the integral term may be provided in equidistant steps, or may be provided based on different, suitably defined step widths. The plurality of integral threshold values and the associated increases in the gain factor of the integral term may be stored in the form of a table or in the form of a function or in any other suitable manner.

As explained above, the controller can be adapted to reduce the gain factor of the integral term when the number of zero crossings of the control difference during the inspiration phase and/or during the expiration phase exceeds the predetermined zero-crossing threshold value. The predetermined zero-crossing threshold value relates to the number of zero crossings during exactly one inspiration phase and/or during exactly one expiration phase.

A zero-crossing may be defined as a crossing of the ventilation variable and the target curve for the ventilation variable. It is also possible that a zero crossing is defined as a crossing of the ventilation variable and the target curve for the ventilation variable in combination with a leaving of a tolerance range defined around the target curve. In other words, a zero crossing may be defined as a change in sign of the control difference or may be defined as reaching a predetermined control difference after a change in sign of the control difference. By defining a tolerance range or defining a predetermined control difference for detecting a zero crossing, it is possible to prevent an oscillation of the ventilation variable around the target curve with low amplitude from being considered as a reason to adjust the gain factor of the integral term. In other words, oscillation of the ventilation variable around the target curve can be considered acceptable as long as the amplitude of this oscillation around the target curve remains low.

According to another embodiment, a plurality of zero-crossing threshold values are provided, and the controller is adapted to reduce the gain factor of the integral term a plurality of times when the number of zero crossings exceeds several of the plurality of zero-crossing threshold values. In this way, the reduction of the gain factor of the integral term can be effected in a particularly targeted manner. In particular, a large reduction in the gain factor of the integral term can take place when there is a large number of zero crossings indicating a large overshoot in the ventilation variable. The plurality of zero-crossing threshold values and the associated reductions in the gain factor of the integral term may be stored in the form of a table or in the form of a function or in any other suitable manner.

According to a further embodiment, the controller is adapted to increase the gain factor of the integral term in feature (i) with a first delta value. The first delta value may be a predetermined first delta value. In particular, the first delta value may be an absolute value for increasing the gain factor of the integral term or may be a percentage value of the initial value of the gain factor of the integral term described above. In the case where a plurality of integral threshold values are provided, the first delta value may be used for increasing the gain factor of the integral term when a respective one of the plurality of integral threshold values is exceeded. However, it is also possible to use a plurality of first delta values in case of a plurality of integral threshold values.

According to a further embodiment, the controller is adapted to reduce the gain factor of the integral term in feature (ii) by a second delta value. The second delta value may be a predetermined second delta value. In particular, the second delta value may be an absolute value for reducing the gain factor of the integral term or may be a percentage value of the initial value of the gain factor of the integral term described above. In the case where a plurality of zero-crossing threshold values are provided, the second delta value may be used for reducing the gain factor of the integral term when a respective one of the plurality of zero-crossing threshold values is exceeded. However, it is also possible to use a plurality of second delta values in case of a plurality of zero-crossing threshold values.

According to a further embodiment, the first delta value is different from the second delta value. In particular, the first delta value can be between one and five times as large as the second delta value. Further in particular, the first delta value can be between 1.5 times and three times as large as the second delta value. In this way, an adjustment of the controller for a faster tracking of the target curve can be achieved by comparatively high first delta values, while unwanted overshoot of the target curve is slowly reduced by comparatively low second delta values. The relative amounts of the first delta value and the second delta value can be used to achieve a relative importance of quickly adjusting the controller to aggressively track the target curve and reducing or preventing overshoot. It is emphasized that the first delta value and the second delta value may also be equal. Furthermore, it is emphasized that the second delta value may also be larger than the first delta value.

According to a further embodiment, the controller is further adapted to adjust the gain factor of the integral term according to the following feature: (iii) reducing the gain factor of the integral term when the instantaneous value for the ventilation variable exceeds a safety threshold value. In this way, it can be prevented that the ventilation variable follows the target curve too aggressively and that overshooting of the target curve leads to potentially dangerous pulmonary pressure in the patient.

According to a further embodiment, the safety threshold value is between 1.5 mbar and 3 mbar above the target curve.

Exemplary embodiments of the invention further comprise a ventilation device, comprising a system for controlling a ventilation variable according to any of the embodiments described above. The additional features, modifications and effects as described above with reference to the system for controlling a ventilation variable of a ventilation device are analogously applicable to the ventilation device.

According to a further embodiment, the ventilation device comprises a connecting member and a patient application piece, wherein the patient application piece is connected to a patient-side end portion of the connecting member. The connecting member may form the connection between the patient application piece and the other components of the ventilation device, such as a compressed air source, an air humidifier, an inspiration valve, an expiration valve, etc. In particular, the patient application piece may be a tube or a respirator mask, depending on the desired method of ventilating the patient. In particular, the connecting member may be a single-lumen or a double-lumen connecting hose and may be constructed depending on the position of the air outlet for the expiration phase.

According to a further embodiment, the sensor is arranged at a ventilation device-side end portion of the connecting member. According to an alternative embodiment, the sensor is arranged in the region of the connection between the connecting member and the patient application piece. According to a further alternative embodiment, a first sensor is arranged at a ventilation device-side end portion of the connecting member and a second sensor is arranged in the region of the connection between the connecting member and the patient application piece. When a single sensor is used, the position can be selected depending on the required accuracy, complexity of positioning, and susceptibility to error. While the region of the connection between the connecting member and the patient application piece is closer to the patient and thus potentially provides a better estimate of the air pressure prevailing in the lungs, positioning the sensor at the ventilation device-side end portion of the connecting member may result in a less complex implementation and/or less susceptibility to interference effects. When two sensors are used, it is possible by comparison of the measured values to detect errors in the ventilation system and/or to perform additional adjustments of the controller on the basis of the dead volume between the sensors.

Exemplary embodiments of the invention comprise furthermore a method for controlling a ventilation variable of a ventilation device for tracking a target curve, said method comprising the steps of: acquiring instantaneous values for the ventilation variable; determining a control difference from the instantaneous values for the ventilation variable and the target curve for the ventilation variable, the target curve being given for an inspiration phase and/or for an expiration phase; determining a control variable for an actuator, wherein determining the control variable is performed according to a control function having at least a proportional term and an integral term; adjusting the integral term of the control function according to at least one of the following features: (i) increasing the gain factor of the integral term when the integral of the control difference for the inspiration phase and/or the expiration phase exceeds a predetermined integral threshold value; (ii) reducing the gain factor of the integral term when a number of zero crossings of the control difference during the inspiration phase and/or during the expiration phase exceeds a predetermined zero-crossing threshold value; and controlling the actuator according to the determined control variable for adjusting the ventilation variable of the ventilation device. The additional features, modifications and effects as described above with reference to the system for controlling a ventilation variable of a ventilation device are analogously applicable to the method for controlling a ventilation variable of a ventilation device.

According to a further embodiment, controlling the actuator is performed according to the determined control variable for adjusting the ventilation pressure or the ventilation volume of the ventilation device.

According to a further embodiment, the actuator comprises a controlled blower and/or a controlled valve, and the method comprises controlling the controlled blower and/or the controlled valve.

According to a further embodiment, the control function consists of a proportional term and an integral term.

According to a further embodiment, the method leaves a gain factor of the proportional term unchanged when the gain factor of the integral term is adjusted.

According to a further embodiment, the method leaves the gain factor of the proportional term generally unchanged in operation.

According to a further embodiment, the method further comprises: setting the gain factor of the integral term to an initial value.

According to a further embodiment, setting the gain factor of the integral term is performed depending on at least one patient parameter, such as age or weight.

According to a further embodiment, setting the gain factor of the integral term to the initial value is performed once at the start of operation or in predetermined intervals.

According to a further embodiment, adjusting the gain factor of the integral term for tracking the target curve is performed for the current inspiration phase or the current expiration phase.

According to a further embodiment, the method further comprises: setting to zero the integral of the control difference at a zero crossing of the control difference.

According to a further embodiment, a plurality of integral threshold values are provided, and adjusting the integral term of the control function comprises: increasing the gain of the integral term a plurality of times when the integral of the control difference exceeds several of the plurality of integral threshold values.

According to another embodiment, a plurality of zero-crossing threshold values are provided, and adjusting the integral term of the control function comprises: reducing the gain factor of the integral term a plurality of times when the number of zero crossings exceeds several of the plurality of zero-crossing threshold values.

According to a further embodiment, adjusting the integral term of the control function comprises: increasing the gain factor of the integral term in feature (i) with a first delta value and/or reducing the gain factor of the integral term in feature (ii) by a second delta value.

According to a further embodiment, the first delta value is different from the second delta value.

According to a further embodiment, adjusting the integral term of the control function comprises: (iii) reducing the gain factor of the integral term when the instantaneous value for the ventilation variable exceeds the target curve by a safety threshold value.

According to another embodiment, the safety threshold value is between 1.5 mbar and 3 mbar.

Exemplary embodiments of the invention comprise furthermore a computer program or a computer program product containing program instructions which, when executed on a data processing system, perform a method according to any of the embodiments described above. In this regard, the individual steps of the method may be initiated by the program instructions and executed by other components or may be executed in the data processing system itself.

### BRIEF DESCRIPTION OF THE DRAWINGS

Further exemplary embodiments of the invention will be described below with reference to the accompanying drawings, wherein:

FIG. 1 shows a ventilation device for mechanically ventilating patients according to an exemplary embodiment of the invention, the ventilation device being provided with a system for controlling a ventilation variable according to an exemplary embodiment of the invention;

FIG. 2 shows a system for controlling a ventilation variable of a ventilation device according to an exemplary embodiment of the invention in a block diagram;

FIG. 3 illustrates the operation of the system of FIG. 2 by way of an exemplary target curve for the ventilation variable and an exemplary course of the instantaneous values for the ventilation variable;

FIG. 4 illustrates the operation of the system of FIG. 2 by way of an exemplary target curve for the ventilation variable and a further exemplary course of the instantaneous values for the ventilation variable;

FIG. 5 illustrates the operation of the system of FIG. 2 by way of an exemplary target curve for the ventilation variable and a further exemplary course of the instantaneous values for the ventilation variable;

FIG. 6 illustrates the operation of the system of FIG. 2 over several ventilation cycles.

### DETAILED DESCRIPTION OF THE INVENTION

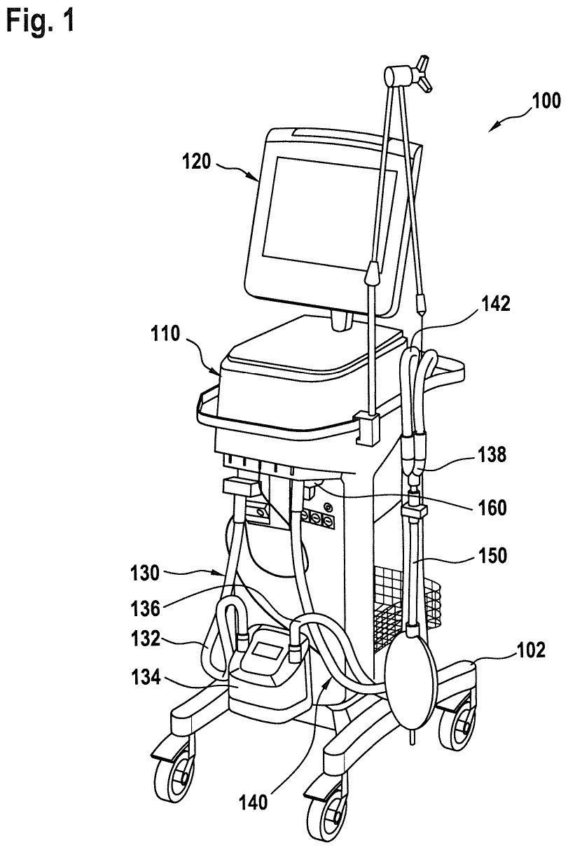

FIG. 1 illustrates a ventilation device 100 for mechanically ventilating patients in accordance with an exemplary embodiment of the invention. The ventilation device 100 has a frame 102 movable on rolls, which is provided with a control unit 110 and a monitor 120 as well as a first line 130 for ventilation air supplied by the device and a second line 140 for exhaled air. The first line 130 for supplied ventilation air includes a first ventilation air hose 132 extending from the control unit 110 and leading to a humidification unit 134, where the ventilation air is passed through a water reservoir. From the humidification unit 134, a second ventilation air hose 136 leads to a T-piece 138. From the T-piece, the ventilation air provided by the ventilation machine 100 during an inspiration phase of the ventilation cycle passes to the patient via a patient application piece 150. The air exhaled by the patient during an expiration phase of the ventilation cycle also passes back toward the ventilation device 100 via the patient application piece 150. For this purpose, another breathing air hose 142 branches off from the T-piece 138, which belongs to the second line 140 for exhaled air. The breathing air hose 142 leads to an expiration valve 160 through which the exhaled air is released into the environment of the ventilation device.

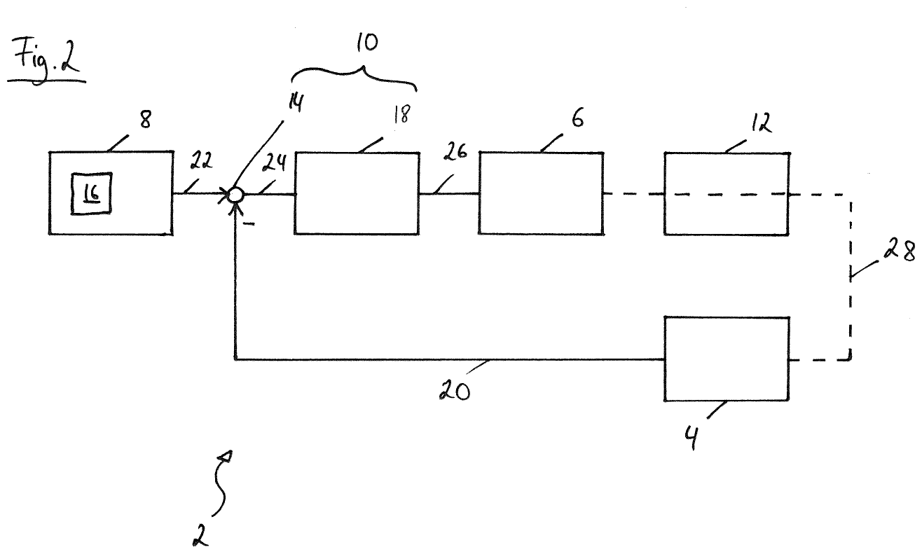

Embedded within the ventilation device 100 is a system for controlling a ventilation variable according to an exemplary embodiment of the invention. The individual components of this system for controlling a ventilation variable will be described below with reference to FIG. 2, and reference will be made again to FIG. 1 with respect to an exemplary positioning of the components.

FIG. 2 shows a system 2 for controlling a ventilation variable of a ventilation device according to an exemplary embodiment of the invention in a block diagram.

The system 2 has a memory 8 in which a target curve 16 for a ventilation variable is stored. In the exemplary embodiment of FIG. 2, the ventilation variable is the ventilation pressure provided by the ventilation device. Accordingly, the target curve 16 is a sequence of ventilation pressure values over time. In particular, the target curve 16 may comprise a sequence of ventilation pressure values over an inspiration phase and/or over an expiration phase of a ventilation cycle. The target curve 16 represents a desired course of the ventilation pressure over time. The target curve 16 is output from the memory 8 as a sequence of nominal or target pressure values. In particular, the target curve 16 is output as a signal 22 from the memory 8, the signal 22 representing an image of the target curve 16.

The system 2 comprises a sensor 4 that is designed to detect the ventilation variable. In the exemplary embodiment of FIG. 2, the sensor 4 is a pressure sensor. The sensor 4 senses the ventilation pressure 28 and outputs instantaneous values 20 for the ventilation pressure. The ventilation pressure 28 is represented as a physical quantity by a dashed line in FIG. 2, whereas the instantaneous values 20 for the ventilation pressure are a signal representation of the physical quantity and are represented by a solid line in FIG. 2.

The signal 22 representing the target curve 16 and the instantaneous values 20 of the ventilation pressure are supplied to a subtractor 14 which forms a control difference 24 from the target curve 16 and the instantaneous values 20 for the ventilation pressure. In doing so, the subtractor 14 in each case subtracts an instantaneous value of the ventilation pressure from a current or instantaneous target value of the target curve. Repeated subtraction results in a time curve of the control difference 24 and thus a time curve of the deviation between the target curve 16 and the instantaneous values 20 of the ventilation pressure.

The subtractor 14 is part of a controller 10 of the system 2. In addition to the subtractor 14, the controller 10 has a control function 18 which can also be referred to as control algorithm 18. The control difference 24 is provided to the control function 18 which is connected to the subtractor 14. A time curve or history of the control difference 24 is provided to the control function 18 over time. The control function 18 uses the time curve of the control difference 24 to generate a control variable 26 and to cause an actuator 6 by means of the control variable 26 to act on the ventilation pressure 28

The controller 10 has a proportional term and an integral term. In the exemplary embodiment of FIG. 2, the controller 10 has only a proportional term and an integral term. Thus, the controller 10 is a PI controller in the exemplary embodiment of FIG. 2. The controller 10 may operate according to the following formula:

$$u ( t ) = {K}_{P} * e ( t ) + {K}_{\text{?}} * {\int }_{\text{?}}^{\text{?}} e ( τ ) d τ ,$$

$$\text{?} \text{indicates text missing or illegible when filed}$$

wherein u(t) designates the control variable 26 and e(t) designates the control difference 24. The linear term before the “+” sign is the proportional term of the controller 10, wherein K_{P} designates the gain factor of the proportional term. The integral term after the “+” sign is the integral term of the controller 10, wherein K_{I} designates the gain factor of the integral term.

It is emphasized that the controller 10 can also operate according to a different formula. However, the controller 10 always has a proportional term that takes into account the current or instantaneous control difference, and an integral term that takes into account the control difference over a certain or determinable time horizon in the past. Thus, the accumulated control difference over this time horizon is considered in the control. The time horizon may comprise a certain period of time or a period of time since a certain event, e.g. the period of time since the beginning of the current ventilation cycle or since the beginning of the current inspiration phase or expiration phase.

The actuator 6 adjusts the ventilation pressure 28 based on the control variable 26. The control variable 26 may indicate to the actuator 6 to increase or decrease the ventilation pressure 28 or keep it the same. Furthermore, the control variable 26 may indicate to the actuator 6 how much the ventilation pressure is to be adjusted.

In the exemplary embodiment of FIG. 2, the actuator 6 is a source of compressed air. For this purpose, the actuator 6 may comprise, for example, a controlled compressed air pump or a controlled blower or a controlled fan. It is also possible that the actuator 6 comprises a compressed air reservoir and a controlled valve. In either case, the actuator 6 is capable of outputting compressed air toward the patient being ventilated in controlled manner.

The actuator 6 is adapted to adjust the ventilation pressure 28. For a given application of compressed air by the actuator 6, very different results in the ventilation pressure 28 may arise. The reason for this is the ventilation tract 12 of the patient being ventilated, which varies from person to person. In particular, the ventilation tract 12 of each patient has a certain resistance with respect to applied compressed air and a certain expansion capacity of the ventilation tract 12 when compressed air is applied. Because of the difference in resistance and expansion capacity of the ventilation tract 12, a patient's response to the compressed air applied by the actuator 6 can vary greatly. These relationships are illustrated in FIG. 2 by showing the ventilation pressure 28 as a dashed line connected to the actuator 6 and extending through the ventilation tract 12. The ventilation pressure 28 is influenced by the compressed air output from the actuator 6 and by the characteristics of the ventilation tract 12. The resulting ventilation pressure 28 is measured by the sensor 4, as illustrated by the connection of said dashed line to the sensor 4.

In the ventilation device 100 of FIG. 1, the memory 8 and the controller 10 may be arranged in the control unit 110. The actuator 6 may also be arranged in the control unit 110. The sensor 4 may be disposed in the ventilation device-side end portion of the first line 130 or in the region of the T-piece 138 or at another suitable location between the control unit 110 and the patient application piece 150. The position of the sensor 4 may be selected depending on requirements for distance between the sensor 4 and the patient and/or depending on requirements for signal transmission between the sensor 4 and the control unit 110. Also, the position of the sensor 4 may depend on whether the control of the ventilation pressure takes place only for the inspiration phase or only for the expiration phase or for the inspiration phase and the expiration phase.

The controller 10 may be implemented in hardware or implemented as software to be executed on a processor, or implemented as a combination of hardware and software. In addition to the control of the actuator 6 described above, the controller 10 of the exemplary embodiment of FIG. 2 has a further functionality affecting the control of the ventilation pressure 28. The controller 10 is adapted to adjust its integral term based on the control difference 24. Thus, the controller 10 is adapted to adjust itself based on the control difference 24. The controller 10 can thereby change the influence on the control of the actuator 6 and the influence on the ventilation pressure 28 based on the control difference. Thus, the control variable 26 is dependent not only on the history of the control difference 24, but also on the adjustment of the control algorithm 18 within the controller 10 based on the history of the control difference 24. In this way, the controller 10 can be very flexible in responding to the circumstances of the ventilation at hand. In particular, by adjusting its own control algorithm 18, the controller 10 can achieve an implicit adjustment of the control to the ventilation tract 12 of the patient to be ventilated.

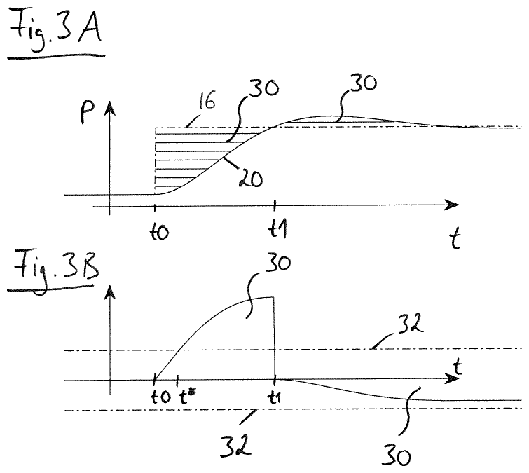

In the exemplary embodiment of FIG. 2, the controller 10 is adapted to increase the gain factor K_{I} of the integral term when the integral of the control difference 24 exceeds a predetermined integral threshold value. Furthermore, the controller 10 is adapted to reduce the gain factor K_{I} of the integral term when a number of zero crossings of the control difference 24 exceeds a predetermined zero-crossing threshold value. Still further, the controller 10 is adapted to reduce the gain factor K_{I} of the integral term when the instantaneous value 20 for the ventilation variable exceeds the target curve 16 by a safety threshold value. In the exemplary embodiment of FIG. 2, all three of the aforementioned criteria for adjusting the gain factor K_{I} of the integral term are implemented. However, it is also possible that only one of the three criteria mentioned or a subset of the criteria mentioned is implemented for adjusting the gain factor K_{I} of the integral term. The three criteria mentioned will be explained in more detail below with reference to FIGS. 3 to 5.

In the exemplary embodiment of FIG. 2, the controller 10 considers each inspiration phase or expiration phase in isolation for adjusting the gain factor K_{I} of the integral term. In other words, the integral of the control difference 24 or the number of zero crossings of the control difference 24 is set to zero at the end of an inspiration phase or at the end of an expiration phase. Furthermore, the integral of the control difference 24 is also set to zero within an inspiration phase or within an expiration phase at a zero crossing of the control difference 24. However, it is also possible that the controller 10 determines the integral of the control difference 24 or the number of zero crossings of the control difference 24 over a longer period of time and adjusts the gain factor K_{I} of the integral term based on this determination.

FIG. 3 shows an exemplary course of the instantaneous values 20 of a ventilation variable for a sudden increase of the target curve 16 of the ventilation variable as well as an associated integral 30 of the control difference. FIG. 3 illustrates the operation of the system 2 of FIG. 2 for an exemplary target curve 16 and an exemplary course of the instantaneous values 20 of the ventilation variable.

In FIG. 3A, the target curve 16 for the ventilation pressure 28 and the course of the instantaneous values 20 for the ventilation pressure 28 are plotted over time. In the illustrative example of FIG. 3A, the target curve 16 makes a positive pressure jump at the time to. The positive pressure jump at the time to is suitable for illustrating the behavior of the controller 10 at the beginning of an inspiration phase. It is apparent to those skilled in the art that the target curve 16 at the beginning of the inspiration phase may have a different increase than the hard jump shown in FIG. 3A.

The controller 10 responds to the pressure jump of the target curve 16 at the time to and acts to track the ventilation pressure 28 so as to follow the target curve 16. FIG. 3A shows an example of a so-called step response of the controller 10. As illustrated in FIG. 3A, the instantaneous values 20 for the ventilation pressure 28 increase only slowly in the example of FIG. 3A, as compared to the step increase of the target curve 16. The controller 10 can track the ventilation pressure 28 so as to follow the target curve 16 only slowly. In FIG. 3A, the integral 30 of the control difference 24, i.e. the integral 30 of the difference between target curve 16 and instantaneous values 20 of the ventilation pressure 28 is illustrated as a hatched area. By the time t1, when the instantaneous value 20 of the ventilation pressure reaches the target curve 16 for the first time, the integral 30 of the control difference 24 has reached a comparatively high value.

In FIG. 3B, the integral 30 of the control difference 24, which is illustrated as a hatched area in FIG. 3A, is plotted as a curve versus time. At the time to, the integral 30 is zero, and at the time t1, the integral 30 is reset to zero. In operation, the integral 30 of the control difference 24 is compared to an integral threshold value 32. Between t0 and t1, there is a time t* at which the integral 30 of the control difference 24 exceeds the integral threshold value 32.

As a result of exceeding the integral threshold value 32, the controller 10 increases the gain factor K_{I} of the integral term. In this way, the controller 10 responds more strongly to a cumulative control difference starting from the time t*. This, in turn, enables faster tracking of the ventilation pressure 28 along the target curve 16 for the future.

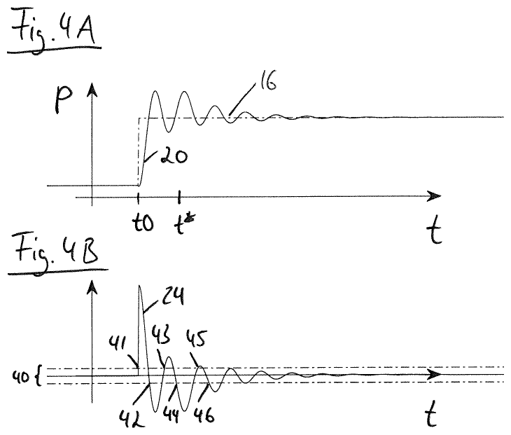

FIG. 4 shows a further exemplary course of the instantaneous values 20 of a ventilation variable for a sudden increase in the target curve 16 of the ventilation variable, as well as an associated course of the control difference. FIG. 4 illustrates the operation of the system 2 of FIG. 2 for an exemplary target curve 16 and a further exemplary course of the instantaneous values 20 of the ventilation variable.

In FIG. 4A, the target curve 16 for the ventilation pressure 28 and the course of the instantaneous values 20 for the ventilation pressure 28 are plotted over time. In the illustrative example of FIG. 4A, the target curve 16 makes a positive pressure jump at the time to. Again, the positive pressure jump at the time to is suitable for illustrating the behavior of the controller 10 at the beginning of an inspiration phase. It is apparent to those skilled in the art that the target curve 16 at the beginning of the inspiration phase may have a different increase than the hard jump shown in FIG. 4A.

The controller 10 responds to the pressure jump of the target curve 16 at the time to and acts to track the ventilation pressure 28 so as to follow the target curve 16. FIG. 4A shows another example of a so-called step response of the controller 10. In contrast to the situation illustrated in FIG. 3A, the controller 10 in the situation of FIG. 4A is able to track the ventilation pressure 28 so as to follow the target curve 16 much faster. The instantaneous value 20 of the ventilation pressure 28 reaches the target curve 16 already shortly after the time to. However, the increase of the ventilation pressure 28 is so fast that the ventilation pressure 28 overshoots the target curve 16. The course of the instantaneous values 20 of the ventilation pressure 28 crosses the target curve very steeply and overshoots the target curve 16 significantly. The controller 10 reacts to the overshooting of the target curve 16 and acts to reduce the ventilation pressure 28. As a result, the course of the instantaneous values 20 of the ventilation pressure 28 overshoots the target curve 16 downward. The attempt of the controller 10 to control the ventilation pressure 28 so as to follow the target curve 16 results in a rather high-frequency oscillation of the course of the instantaneous values 20 of the ventilation pressure 28 around the target curve 16.

FIG. 4B shows the control difference 24, i.e. the difference between target curve 16 and instantaneous values 20 of the ventilation pressure 28, versus time. Furthermore, FIG. 4B shows a tolerance range 40 for the control difference 24 around the value 0. In the exemplary embodiment of FIG. 4, a zero crossing of the control difference 24 is defined as starting from or crossing the value zero and then leaving the tolerance range 40. Based on this definition, the exemplary control difference 24 of FIG. 4 involves six zero crossings 41, 42, 43, 44, 45, and 46.

In the exemplary embodiment of FIG. 4, a zero-crossing threshold value of three is provided. At the time t*, as illustrated in FIG. 4A, the control difference 42 passes through the fourth zero crossing 44 and has thus exceeded the zero-crossing threshold value of three. It is also possible that the concept of exceeding the zero-crossing threshold value includes reaching the zero-crossing threshold value. In this case, the time t* would already be at the time of the third zero crossing 43.

As a consequence of exceeding the zero-crossing threshold value, the controller 10 reduces the gain factor K_{I} of the integral term. In this manner, the controller 10 responds less strongly to a cumulative control difference starting from the time t*. This, in turn, allows slower tracking of the ventilation pressure 28 along the target curve 16 for the future, and thus less overshooting and oscillation around the target curve 16.

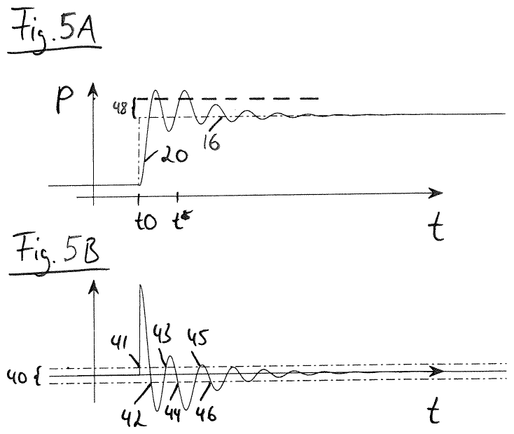

FIG. 5 shows a further exemplary course of the instantaneous values 20 of a ventilation variable for a sudden increase in the target curve 16 of the ventilation variable as well as an associated course of the control difference. FIG. 5 illustrates the operation of the system 2 of FIG. 2 for an exemplary target curve 16 and a further exemplary course of the instantaneous values 20 of the ventilation variable.

FIG. 5 is identical to FIG. 4 except that a safety threshold value 48 is provided in addition to the target curve 16 and in addition to the course of the instantaneous values 20 of the ventilation variable. In the exemplary embodiment of FIG. 5, the safety threshold value 48 is defined as an incremental value with respect to the target curve 16. The safety threshold value may also be defined as an absolute value or in any other suitable manner. The controller 10 is adapted to reduce the gain factor K_{I} of the integral term when the course of the instantaneous values 20 exceeds the safety threshold value 48, i.e. when the course of the instantaneous values 20 exceeds the target curve 16 by more than the safety threshold value 48. In this way, overshooting of the instantaneous values 20 of the ventilation pressure above the target curve 16 can be reduced and reaching of potentially dangerous values for the ventilation pressure can be rendered more difficult or prevented.

In the exemplary course of the instantaneous values 20 of FIG. 5, the safety threshold value 48 is exceeded twice. Consequently, the gain factor K_{I} of the integral term is reduced twice as a result of the safety threshold value 48 being exceeded and once as a result of the zero-crossing threshold value being exceeded. Thus, a total of three reductions of the gain factor K_{I} of the integral term take place.

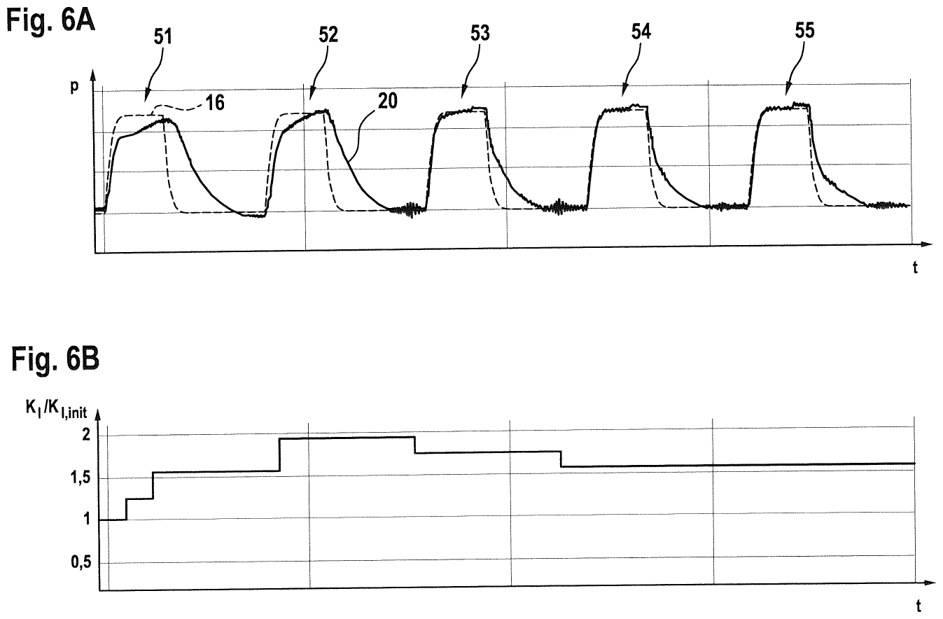

FIG. 6 shows a further exemplary course of the instantaneous values 20 of a ventilation variable for a further exemplary target curve 16 of the ventilation variable. FIG. 6 shows the course of the instantaneous values 20 as well as the target curve 16 for a plurality of ventilation cycles and shows the associated adjustment of the gain factor of the integral term of the controller. FIG. 6 illustrates the operation of the system 2 of FIG. 2 for another exemplary target curve 16 and another exemplary course of the instantaneous values 20 of the ventilation variable.

FIG. 6A shows five ventilation cycles 51, 52, 53, 54, and 55, each of which has an inspiration phase and an expiration phase. The target curve 16 represents a good approximation to a target curve as it is used in the actual ventilation of a patient. In the inspiration phase, the target ventilation pressure first rises sharply and then remains at a plateau. In the expiration phase, the target ventilation pressure first drops sharply and then remains at the so-called positive end-expiratory pressure (PEEP). The positive end-expiratory pressure remains applied to the patient to prevent collapse of the lungs or individual alveoli of the lungs.

In the exemplary embodiment of FIG. 6, the controller 10 operates to track the instantaneous values 20 of the ventilation pressure 28 so as to follow the target curve 16 in the inspiration phase as closely as possible. In the expiration phase, a passive release of the ventilation pressure 28 takes place first, i.e. the controller 10 does not support the reduction of the ventilation pressure 28 in an active manner. The course of the instantaneous values 20 of the ventilation pressure 28 decreases more slowly than the target curve 16. In the further course of the expiration phase, the controller 10 acts to keep the ventilation pressure 28 at the positive end-expiratory pressure.

FIG. 6B illustrates the gain factor of the integral term of the controller 10 over time. The gain factor is plotted in FIG. 6B as a quantity normalized to the initial value of the gain factor. In other words, FIG. 6B shows along the y axis the dimensionless value for the quotient of instantaneous gain factor of the integral term to initial value of the gain factor of the integral term, i.e. the dimensionless value for the quotient K_{I}/K_{I,init}.

In the inspiration phase of the first ventilation cycle 51, there is present a relatively high value for the integral of the control difference 24. This is evident from the relatively large area between the target curve 16 and instantaneous values 20 for the ventilation pressure 28. In the exemplary embodiment of FIG. 6, the gain factor of the integral term is increased twice due to this relatively high value for the integral of the control difference 24. This two-fold increase in the gain factor of the integral term corresponds to two integral threshold values being exceeded. That is, the exemplary embodiment of FIG. 6 has a plurality of integral threshold values, and when a respective one of the plurality of integral threshold values is exceeded, there is taking place an increase in the gain factor of the integral term. Each increase occurs by 0.3 on the normalized scale of FIG. 6B.

In the inspiration phase of the second ventilation cycle 52, the controller 10 is able to better track the instantaneous values 20 of the ventilation pressure 28 to follow the target curve 16. However, the integral of the control difference 24 also has a significant value in the inspiration phase of the second ventilation cycle 52. This value is above the lowest integral threshold value, so that in the inspiration phase of the second ventilation cycle 52 there is taking place a further increase of the gain factor of the integral term. This increase also occurs by 0.3 on the normalized scale of FIG. 6B.

In the expiration phase of the second ventilation cycle 52, the course of the instantaneous values 20 of the ventilation pressure 28 oscillates around the target curve 16, in particular around the positive end-expiratory pressure. There is a number of zero crossings occurring that is greater than the predetermined zero-crossing threshold value. In the exemplary embodiment of FIG. 6, a zero-crossing is defined similarly to FIG. 4B and FIG. 5B, namely as an overshoot over a predetermined tolerance range about the positive end-expiratory pressure. It is evident from FIG. 6A that the instantaneous values 20 of the ventilation pressure 28 oscillate comparatively strongly about the target curve 16 during the expiration phase of the second inspiration cycle 52. As a result, the predetermined zero-crossing threshold value is exceeded. In the exemplary embodiment of FIG. 6, the gain factor of the integral term is reduced due to the predetermined zero-crossing threshold value being exceeded. The reduction is by 0.15 on the normalized scale of FIG. 6B.

In the inspiration phase of the third ventilation cycle 53, the integral of the control difference is so small that no adjustment is made to the gain factor of the integral term. In the expiration phase of the third ventilation cycle 53, the course of the instantaneous values 20 of the ventilation pressure 28 again oscillates around the target curve 16 such that the number of zero crossings exceeds the predetermined zero-crossing threshold value. Again, the gain factor of the integral term is reduced by 0.15 on the normalized scale of FIG. 6B.

In the fourth ventilation cycle 54 and the fifth ventilation cycle 55, the controller 10 tracks the ventilation pressure to follow the target curve 16 so well that no further adjustment is made to the gain factor of the integral term. The controller has adapted itself so well to the conditions of the present ventilation situation over the course of the first through third ventilation cycles 51 to 53 that an effective tracking of the ventilation pressure 28 along the target curve is possible.

Although the invention has been described with reference to exemplary embodiments, it is apparent to one skilled in the art that various modifications may be made and equivalents used without departing from the scope of the invention. The invention is not intended to be limited by the specific embodiments described. Rather, it includes all embodiments covered by the appended claims.

1. A system for controlling a ventilation variable of a ventilation device for tracking a target curve, comprising:
at least one sensor which is adapted to detect instantaneous values for the ventilation variable;
an actuator which is adapted to adjust the ventilation variable of the ventilation device;
a memory in which the target curve for the ventilation variable is stored, the target curve being stored for an inspiration phase and/or for an expiration phase; and
a controller which has at least a proportional term and an integral term and which is adapted to determine a control difference from the target curve and the instantaneous values for the ventilation variable, and which is adapted to control the actuator;
wherein the controller is adapted to adjust a gain factor of the integral term according to at least one of the following features:
(i) increasing the gain factor of the integral term when the integral of the control difference for the inspiration phase and/or the expiration phase exceeds a predetermined integral threshold value;
(ii) reducing the gain factor of the integral term when a number of zero crossings of the control difference during the inspiration phase and/or during the expiration phase exceeds a predetermined zero-crossing threshold value.

2. The system according to claim 1,
wherein the ventilation variable is one of ventilation pressure and ventilation volume.

3. The system according to claim 1,
wherein the actuator comprises a controlled blower and/or a controlled valve.

4. The system according to claim 1,
wherein the controller is a proportional integral controller.

5. The system according to claim 1,
wherein the controller is adapted to leave a gain factor of the proportional term unchanged when the gain factor of the integral term is adjusted.

6. (canceled)

7. The system according to claim 1,
wherein the controller is adapted to set the gain factor of the integral term to an initial value.

8. The system according to claim 7,
wherein the initial value is dependent on at least one patient parameter, such as age or weight.

9. The system according to claim 7,
wherein the controller is adapted to set the gain factor of the integral term to the initial value once at the start of operation, or is adapted to set the gain factor of the integral term to the initial value in predetermined intervals.

10. The system according to claim 1,
wherein the controller is adapted to adjust the gain factor of the integral term for tracking the target curve for the current inspiration phase or the current expiration phase.

11. The system according to claim 1,
wherein the controller is adapted to set the integral of the control difference to zero at a zero crossing of the control difference.

12. The system according to claim 1,
wherein a plurality of integral threshold values are provided and wherein the controller is adapted to increase the gain factor of the integral term a plurality of times when the integral of the control difference exceeds several of the plurality of integral threshold values.

13. The system according to claim 1,
wherein a plurality of zero-crossing threshold values are provided, and wherein the controller is adapted to reduce the gain factor of the integral term a plurality of times when the number of zero crossings exceeds several of the plurality of zero-crossing threshold values.

14. The system according to claim 1,
wherein the controller is adapted to increase the gain factor of the integral term in feature with a first delta value, and/or wherein the controller is adapted to reduce the gain factor of the integral term in feature by a second delta value.

15. (canceled)

16. The system according to claim 1,
wherein the controller is further adapted to adjust the gain factor of the integral term according to the following feature:
(ii) reducing the gain factor of the integral term when the instantaneous value for the ventilation variable exceeds the target curve by a safety threshold value.

17. The system according to claim 16,
wherein the safety threshold value is between 1.5 mbar and 3 mbar.

18. A ventilation device, comprising a system for controlling a ventilation variable according to claim 1.

19. The ventilation device according to claim 18, further comprising:
a connecting member, in particular a single-lumen or double-lumen connecting hose, and
a patient application piece, in particular a tube or a respiratory mask, wherein the patient application piece is connected to a patient-side end portion of the connecting member.

20. The ventilation device according to claim 19,
wherein the sensor is arranged at a ventilation device-side end portion of the connecting member or wherein the sensor is arranged in the region of the connection between the connecting member and the patient application piece or wherein a first sensor is arranged at a ventilation device-side end portion of the connecting member and a second sensor is arranged in the region of the connection between the connecting member and the patient application piece.

21. A method for controlling a ventilation variable of a ventilation device for tracking a target curve, comprising the steps of:
acquiring instantaneous values for the ventilation variable;
determining a control difference from the instantaneous values for the ventilation variable and the target curve for the ventilation variable, the target curve being given for an inspiration phase and/or for an expiration phase;
determining a control variable for an actuator, wherein determining the control variable is performed according to a control function having at least a proportional term and an integral term;
adjusting the integral term of the control function according to at least one of the following features:
(i) increasing the gain factor of the integral term when the integral of the control difference for the inspiration phase and/or the expiration phase exceeds a predetermined integral threshold value;
(ii) reducing the gain factor of the integral term when a number of zero crossings of the control difference during the inspiration phase and/or during the expiration phase exceeds a predetermined zero-crossing threshold value; and
controlling the actuator according to the determined control variable for adjusting the ventilation variable of the ventilation device.

22. A computer program containing program instructions which, when executed on a data processing system, perform a method according to claim 21.
